(19) United States
(12) Patent Application Publication
(10) Pub. No.: US 2019/0179037 A1
(43) Pub. Date: Jun. 13, 2019

(54) SYSTEM AND METHOD FOR DETERMINING QUANTITIES OF RADON IN AN ENVIRONMENT

(71) Applicant: Joseph DiCianni, Elmhurst, IL (US)

(72) Inventor: Joseph DiCianni, Elmhurst, IL (US)

(21) Appl. No.: 16/218,254

(22) Filed: Dec. 12, 2018

Related U.S. Application Data
(60) Provisional application No. 62/597,679, filed on Dec. 12, 2017.

(51) Int. Cl. G01T 1/178; G01N 33/00

(52) U.S. Cl. CPC G01T 1/178; G01N 2033/0068; G01N 33/0027; G01N 33/0063

(57) ABSTRACT

A system for monitoring a level of gas comprises a gas testing device, an adapter, a mobile device, and a server. The gas testing device comprises a sensor and a processor. The sensor collects a measurement of gas in a location. The processor receives a signal including the measurement, generates data based on the signal, and stores the data in memory. The adapter is coupled to the gas testing device by a serial port and comprises an adapter processor and an adapter memory. The adapter processor receives the data and stores the data in the adapter memory. The mobile device is coupled to the adapter and receives the data from the adapter. The server is coupled to the mobile device via a network and comprises a server processor and a server memory. The server processor receives the data from the mobile device and stores the data in the server memory.

### CROSS-REFERENCE TO RELATED APPLICATIONS

This application claims the benefit of U.S. Provisional Patent Application No. 62/597,679, filed Dec. 12, 2017, which is hereby incorporated by reference in its entirety.

### TECHNICAL FIELD

This disclosure is related to gas detection systems and methods. More specifically, this disclosure is related to a system and associated method for determining quantities of radon in an environment.

### SUMMARY OF THE DISCLOSURE

One embodiment of this disclosure relates to a system for monitoring a level of gas comprises a gas testing device, an adapter, a mobile device, and a server. The gas testing device comprises a sensor and a processor. The sensor collects a measurement of gas in a location. The processor receives a signal including the measurement, generates data based on the signal, and stores the data in memory. The adapter is coupled to the gas testing device by a serial port and comprises an adapter processor and an adapter memory. The adapter processor receives the data and stores the data in the adapter memory. The mobile device is coupled to the adapter and receives the data from the adapter. The server is coupled to the mobile device via a network and comprises a server processor and a server memory. The server processor receives the data from the mobile device and stores the data in the server memory.

Yet another embodiment of this disclosure relates to a method for monitoring a level of gas. The method comprises the steps of collecting, by at least one sensor of a gas testing device, a measurement of an amount of gas in a location; receiving, by a processor of the gas testing device, a signal from the at least one sensor, wherein the signal includes the measurement, generating, by the processor of the gas testing device, data based on the signal; storing, by the processor of the gas testing device, the data in a memory; receiving, by an adapter processor of an adapter coupled to the gas testing device by a serial port of the gas testing device, the data from the gas testing device; storing, by the adapter processor, the data in an adapter memory of the adapter; receiving, by a mobile device coupled to the adapter, the data from the adapter; receiving, by a server processor of a server coupled to the mobile device via a network, the data from the mobile device; and storing, by the server processor, the data in a server memory of the server.

### BRIEF DESCRIPTION OF THE DRAWINGS

Referring now to the drawings, exemplary illustrations are shown in detail. Although the drawings represent examples, the drawings are not necessarily to scale and certain features may be exaggerated or schematic in form to better illustrate and explain a particular aspect of an illustrative example. Any one or more of these aspects can be used alone or in combination within one another. Further, the exemplary illustrations described herein are not intended to be exhaustive or otherwise limiting or restricting to the precise form and configuration shown in the drawings and disclosed in the following detailed description. Exemplary illustrations are described in detail by referring to the drawings as follows:

FIG. 1 is a block diagram of a system for monitoring levels of radon or other gases within a building;

FIG. 2 is a block diagram of an exemplary adapter that may be used with the system shown in FIG. 1; and

FIG. 3 is a flowchart illustrating a method of generating a radon testing report according to one embodiment.

### DETAILED DESCRIPTION

In the following description, numerous specific details are set forth in order to provide a thorough understanding of the present invention. It will be apparent, however, to one having ordinary skill in the art that the specific detail need not be employed to practice the present invention. In other instances, well-known materials or methods have not been described in detail in order to avoid obscuring the present invention.

Reference throughout this specification to “one embodiment”, “an embodiment”, “one example” or “an examples” means that a particular feature, structure or characteristic described in connection with the embodiment of example is included in at least one embodiment of the present invention. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

Embodiments in accordance with the present invention may be embodied as an apparatus, method, or computer program product. Accordingly, the present invention may take the form of an entirely hardware embodiment, an entirely software embodiment (including firmware, resident software, micro-code, etc.), or an embodiment combining software and hardware aspects that may all generally be referred to herein as a “module” or “system”. Furthermore, the present invention may take the form of a computer program product embodied in any tangible media or expression having computer-usable program code embodied in the media.

Any combination of one or more computer-usable or computer-readable media (or medium) may be utilized. For example, a computer-readable media may include one or more of a portable computer diskette, a hard disk, a random access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of the present invention may be written in any combination of one or more programming languages.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisional via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

The flowchart and block diagram(s) in the flow diagram(s) illustrate the architecture, functionality, and operation of possible implementations of systems, methods, and computer program products according to various embodiments of the present invention. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware-based systems that perform the specified functions or acts, or combinations of special purpose hardware and computer instructions. These computer program instructions may also be stored in a computer-readable media that can direct a computer or other programmable data processing apparatus to function in a particular manner, such that the instructions stored in the computer-readable media produce an article of manufacture including instruction means which implement the function/act specified in the flowchart and/or block diagram block or blocks.

Several (or different) elements discussed below, and/or claimed, are described as being “coupled”, “in communication with” or “configured to be in communication with”. This terminology is intended to be non-limiting, and where appropriate, be interpreted to include without limitation, wired and wireless communication using any one or a plurality of suitable protocols, as well as communication methods that are constantly maintained, are made on a periodic basis, and/or made or initiated on an as needed basis.

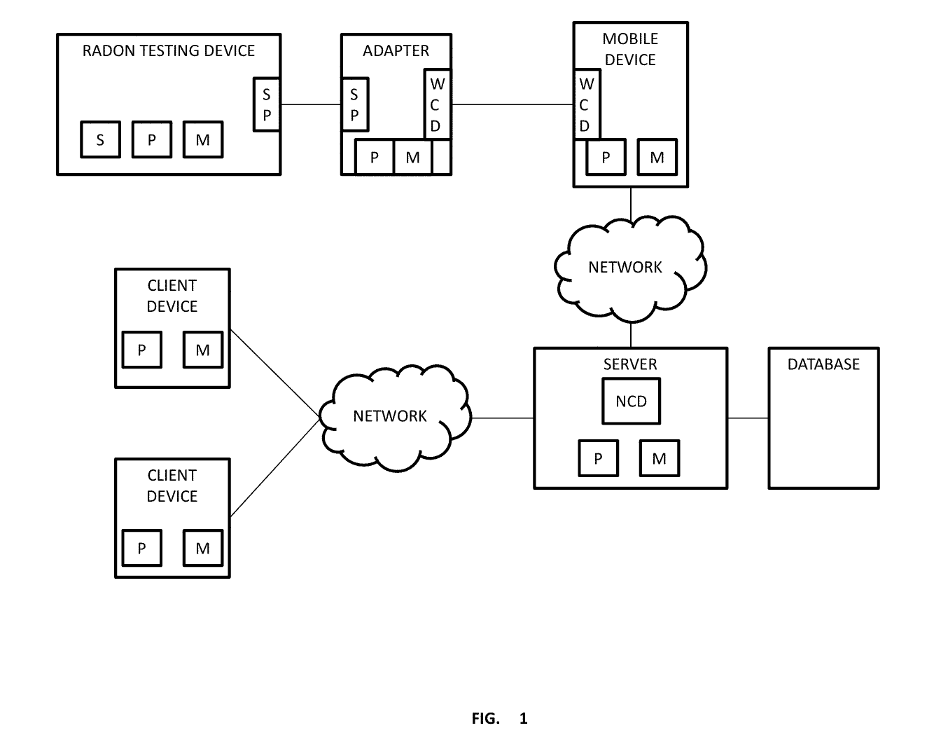

FIG. 1 is a block diagram of a system for monitoring levels of radon or other gases within a building. In the embodiment shown in FIG. 1, the system includes a radon testing device, an adapter coupled to the radon testing device, and a mobile device coupled to the adapter. The system also includes a server coupled to the mobile device via a network, and a database coupled to the server. One or more client devices may also be coupled to the server via a network. While the embodiments described herein include a radon testing device, it should be recognized that the system may include any suitable device for testing any suitable gas or particulate within a building. For example, the system may be used with smoke detectors, carbon monoxide detectors, asbestos detectors, mold detectors, and/or any other suitable detector.

The radon testing device includes at least one sensor (S) for measuring the amount of radon gas present in the ambient air within the building or other location in which the radon testing device is placed. A processor (P) receives a measurement signal from the sensor and generates measurement data from the signal. The processor may store the measurement data in a memory (M) within the radon testing device. When the processor detects that a suitable device (such as the adapter described herein) is connected to a serial port (SP) of the radon testing device, the processor formats the measurement data into a serial stream of data that is then transmitted to the connected device.

The adapter is designed to physically attach to the radon testing device. Alternatively, the functionality of the adapter may be incorporated within the radon testing device itself in some embodiments. In one embodiment, the adapter is able to be physically connected to the serial port of the radon testing device to receive the measurement data from the radon testing device. For example, a portion of the adapter's serial port may be insertable into a portion of the serial port of the radon testing device, or vice versa, for receiving the data from the radon testing device and for transmitting instructions or commands to the radon testing device. Alternatively, the physical connection may be accomplished by plugging one end of a serial cable into the serial port of the radon testing device and the other end of the serial cable into a serial port (SP) of the adapter. In one embodiment, the serial ports of the radon testing device and the adapter are configured to communicate using the RS-232 data protocol. Alternatively, the serial ports may be configured to communicate using any other suitable serial data protocol, such as USB, Firewire, etc. Accordingly, the adapter may be used to retrofit older generations of radon testing devices to enable the radon testing devices to communicate with other devices in an efficient and convenient manner.

In one embodiment, the adapter may include internal memory (M) for storing the measurement data received from the radon testing device. While the embodiments of the adapter are described herein as using the adapter as a pass-through device that transmits the data from the radon testing device to the mobile device while the adapter is plugged into the radon testing device, it should be recognized that the adapter may store the data for later transmission to the mobile device in some embodiments. For example, a processor (P) of the adapter may receive the data from the radon testing device and may store the data in the adapter's memory. The adapter may then be unplugged from the radon testing device. At a later time, the adapter's processor may be used to transmit the data to the mobile device (or to a device that is not typically mobile, such as a desktop computer).

In one embodiment, the adapter includes a wireless communication device (WCD) for transmitting the measurement data to the mobile device. The wireless communication device may include an adapter and associated antenna that may communicate with the mobile device using WiFi, Bluetooth, Zigbee, 3G, 4G, 5G, or another suitable wireless communication protocol.

In one embodiment, the adapter is powered by one or more internal batteries (not shown). The batteries may be removable such that they may be replaced when depleted of charge, and/or the batteries may be rechargeable. The batteries may be lithium ion batteries, nickel cadmium batteries, lead acid batteries, or any suitable type of battery. Alternatively, in some embodiments, the adapter may receive power through the serial connection to the radon testing device.

In one embodiment, the adapter transmits the measurement data to the mobile device and/or server without reformatting the measurement data. In such an embodiment, the mobile device and/or server may reformat the data as described herein. Alternatively, the adapter may reformat the measurement data received from the radon testing device in some embodiments. For example, the adapter may receive the raw measurement data from the radon testing device and may format the data into three columns of tabulated data for easier readability. The adapter may also parse the raw data and may discard portions of the data. For example, the adapter may discard the first 12 hours of measurement data in an extended test and may compute the average radon levels for the remaining period of time in the extended testing period. In another example, 24 hours, 36 hours, or any other suitable amount of the raw measurement data may be discarded. The remaining measurement data may then be averaged to provide a “closed building extended average”. In one embodiment, the hourly data may be discarded as long as there are 48 continuous data points (i.e., hourly radon measurements) remaining in the measurement data.

The mobile device is a cellular phone in some embodiments. In other embodiments, the mobile device is a tablet computing device, a laptop computer, or any other suitable device. In still other embodiments, the mobile device may be a device that is not be conventionally understood as being mobile, and may thus include a desktop computer or the like. The mobile device includes a processor (P) and a memory (M) for communicating with the adapter and server. The mobile device may communicate with the server via a network, such as the Internet or another suitable local area network (LAN) or wide area network (WAN). More specifically, the processor of the mobile device may use the wireless communication device to transmit data to the server via the network.

In one embodiment, the mobile device executes an application or “app” that is configured to communicate with the server. In a specific embodiment, when the application is launched by a user, the application wirelessly detects the adapter. If any pre-existing wireless credentials are stored on the adapter, the application may erase those credentials. The application then receives the measurement data from the adapter and may reformat the data into a format suitable for transmission to the server. The application may include additional data and may package the additional data with the measurement data to be transmitted to the server. The additional data may include, for example, a name and address of the manufacturer or provider of the adapter, a name and address of the company and/or personnel conducting the radon testing, a date and time that the adapter is connected to the radon testing device, a date and time that the adapter received the measurement data from the radon testing device, and/or any other suitable data. The additional data may be appended to the measurement data or may be included within one or more packets of data transmitted concurrently with the measurement data.

The server includes a processor (P) and internal memory (M). The processor controls a network communication device (NCD) to communicate with the mobile device (e.g., with the application executing on the mobile device). For example, the server may use the network communication device to receive the measurement data from the mobile device as well as any additional data associated with the measurement data. The network communication device may include a network adapter configured to communicate using one or more network protocols, such as wired Ethernet (IEEE 802.3), wireless Ethernet or WiFi (IEEE 802.11), and/or any other suitable protocol. In one embodiment, the server is coupled to a database for storing the measurement data and/or the additional data associated with the radon testing device illustrated in FIG. 1 as well as from other radon testing devices. While one server is illustrated and described herein with reference to FIG. 1, it should be recognized that the functionality attributed to the server may be apportioned among a plurality of servers that are connected together via one or more networks or communication links.

In another embodiment, the mobile device may act as a wireless “hotspot” using Wi-Fi tethering or a similar network sharing technology. For example, the adapter may wirelessly connect to the mobile device, and the mobile device may enable the adapter to connect to a wireless network or the Internet, for example, using the mobile device's wireless communication device. In this example, the mobile device may upload the measurement data from the adapter to the server and/or database (or another suitable database, server, or device). In a specific embodiment, the mobile device will transmit the measurement data to the database and/or server via a first webpage and the server may display the data to users via a second webpage. In such an embodiment, the mobile device may not use the application described above, but may rather act as a pass-through device for enabling the adapter to transmit the measurement data directly to the server and/or database using the mobile device's wireless network connection.

In one embodiment, the server may reformat the data received from the adapter and/or mobile device. For example, the server may receive the measurement data from the adapter and/or mobile device and may format the data into three columns of tabulated data for easier readability. The server may also parse the data and may discard portions of the data. For example, the server may discard the first 12 hours of measurement data in an extended test and may compute the average radon levels for the remaining period of time in the extended testing period. In another example, 24 hours, 36 hours, or any other suitable amount of the raw measurement data may be discarded. The remaining measurement data may then be averaged to provide a “closed building extended average”. In one embodiment, the server may discard hourly data as long as there are 48 continuous data points (i.e., hourly radon measurements) remaining in the measurement data. In addition, the server may display the discarded hours or data points, with the discarded hours or data points highlighted in red or otherwise contrasted with the remaining hours that are included in the computed average.

The database may store the measurement data from each radon testing device as received from the adapter and/or mobile devices described herein. The database may store the measurement data in a job or booking record associated with each radon testing appointment booked by a client. For example, the database may store a separate record for each booking, and each record will include the measurement data received from the radon testing device(s) used to perform the radon testing for that booking, as well as any other additional data associated with the booking. The records may be sorted or arranged using the serial number of the radon testing device and/or may be sorted or arranged by a unique number associated with each booking.

In one embodiment, the server may operate as, or may include, a web server for providing access to the measurement data to one or more client devices that connect to the web server. The web server may provide access to the measurement data and other features of the server via a web application that may be accessed by the client devices. Each client device may be required to provide sufficient authentication data, such as a valid username and password, to access the web server and its associated data.

The server may communicate with the client devices via a network, such as the Internet or another suitable local area network (LAN) or wide area network (WAN). For example, the processor may control the network communication device to communicate with the client devices via the network.

The client device(s) may include one or more mobile cellular phones, tablet computing devices, laptop computers, desktop computers, and/or any other suitable device that enables a user to remotely access data provided by the server. Each client device may include a processor (P) and a memory (M) for processing and storing data received from the web server, for example. In one embodiment, each client device may receive (from the server) a customized report of the measurement data gathered by one or more radon testing devices from a building. The reports may be generated in a format optimized for viewing on a mobile device (hereinafter referred to as a “mobile version”) or for viewing on a desktop computer (hereinafter referred to as a “desktop version”).

Table 1 identifies a list of parties that may be programmed into the server for use in generating a report of the testing of radon within a building.

TABLE 1

```
  1. Property
      a. Address
      b. Lockbox
      c. Occupied
      d. Vent Status
      e. etc
  2. Client (Buyer)
      a. Profile (Name, Email, Phone, Address)
      b. Additional Contacts
  3. Inspector
      a. Profile (Name, Email, Phone, Company)
      b. Additional Contacts
      c. Custom Branding - yes/no
      d. Logo
      e. Signature
  4. Buyer Agent
      a. Profile (Name, Email, Phone, Company)
      b. Additional Contacts
  5. Listing Agent
      a. Profile (Name, Email, Phone, Company)
      b. Additional Contacts
  6. Seller
      a. Profile (Name, Email, Phone)
      b. Additional Contacts
  7. Mitigation Firm
      a. Profile (Company, Owner, License Number,
      Tag Serial Number, Phone, Email)
  8. Attorney
      a. Profile (Name, Email, Phone, Company)
  9. Contact
      a. Parent User
      b. Nickname
      c. Profile
10. Monitor
      a. Serial
      b. Model
      c. Calibration Factor
      d. Date Calibrated
      e. Next Calibration Due
11. RS232 Adapter
      a. Serial
      b. Paid Subscriber ID
12. Room
      a. Floor Level
      b. Room Use
      c. Foundation Type
13. Monitor Placement
      a. Serial
      b. Floor Level
      c. Room ID
      d. Location
      e. Foundation Type
      f. Side-by-Side
      g. Appendix E - yes/no
14. Test
      a. Raw Data (from device)
      b. Device Serial
      c. Adapter Serial
      d. Start Date/Time
      e. End Date/Time
      f. Report Date
      g. Placed By
      h. Retrieved By
      i. Mitigation Company ID
      j. Date of Installation
      k. Status of Radon System
15. Closed Building Form/Non-Interference Agreement
      a. Job ID
      b. Date Sent
      c. Date Signed
      d. Status
      e. etc
16. Employee
      a. Profile (Name, State License Number)
      b. Payout (Percentage, fixed value)
17. Invoice/Invoice Items
      a. Service Description
      b. Amount Billed
      c. Discount
      d. Tax
18. Transactions
      a. Transaction ID
      b. Invoice ID
      c. Transaction Amount
      d. Gateway Type (CC, Offline, etc)
19. Notifications
      a. Report ID
      b. Link to staff profile
      c. Opened Timestamp
```

In one embodiment, administrative access may be granted to a user when the user logs into the server with a valid username and password (or other valid authentication data). The administrative access may present a plurality of windows or tabs to the user to enable the user to navigate to the window or tab with the desired functionality exposed. The windows or tabs may include a monitor window, an employee's window, a spreadsheet export window, and/or a statistical report window. The monitor window may enable the user to add/edit/delete monitors and calibration factors, where the calibration date may be synced with a suitable calendaring program (e.g., Google/Outlook Calendar). The employees window may enable the user to add/edit/delete users, set up their salary/commission model, and select administrative permissions like “Add/Edit Monitors”, “View All Users Timetable”, “Transfer jobs to other users”, etc. The spreadsheet export window may enable the user to generate a spreadsheet of abbreviated data that is formatted to comply with the requirements of a selected state (e.g. the State of Illinois). The statistical report window may enable the user to download and/or view the following reports: total jobs, average radon levels and average radon levels YTD, agents most worked with, inspectors most worked with, elevated vs passed jobs, areas with the most elevated jobs, search average radon levels or probability of being elevated by zip code (public access), and/or filters for selecting date range for calculating statistics (Jan. 1, 2017 to Dec. 31, 2017, etc.).

The server may display a dashboard to the user when the user logs into the server. The dashboard may include a calendar view showing the preview of next seven days with each of the reports listed under each day with a colored tag showing what is the next task for that report. For example, Table 2 illustrates a calendar view that may be presented in some embodiments.

TABLE 2

| | | | | | |
|---|---|---|---|---|---|
| 123 Anystreet | Picked | 123 Anystreet | Dropped | 38 Somestreet | Pending |
| 843 Anystreet | Dropped | 456 Somestreet | Pending | 842 Anystreet | Dropped |
| 26 Somestreet | Pending | 980 Anystreet | Picked | 123 Anystreet | Picked |

The dashboard will also have filters for narrowing down the results based on their location, status, etc., and the user can also switch between the calendar view and a list view. The user will be able to rearrange the reports on the desktop version as well as on the mobile version. The option to assign/transfer a task will also be available in the form of a drag-and-drop bin. The following details may be displayed along with each task: a) General Inspection Date, b) Test Start Date, and c) Status.

The server may use a payment processing system (e.g., USAePay) for payment of invoices by client users (sometimes referred to as clients). While the USAePay system is one example of a payment processing system that may be used, it should be recognized that any suitable payment processing system or gateway may be used. The server may prohibit the client users from viewing their reports unless the payment is made and a lock sign will be displayed to the user in case of non-payment. Once an invoice is paid, the server will disburse commissions (fixed or percentage) to the users that have worked on that particular job and at the end of the month their payroll will be calculated. If the API used by the payment processing system supports storing credit card information on their server for a longer term, the credit card information for repeat clients may not need to be reentered for every job that they order. Rather, the server may bill the credit card that is already stored.

For compliance with certain state-imposed rules and regulations, as well as requirement with other state rules and regulations, the server may generate an Excel workbook containing a list of reports with its data in abbreviated form as required and as given in the sample report by the state. The user will be able to generate reports filtered by a specific date range so that it may also be used for internal audit other than just fulfilling state requirements.

All the report notes and dates (drop-off, pick-up, etc.) will be synced with a selected calendar program (e.g., Google Calendar or Outlook Calendar). Each user will have his or her own work calendar in which their jobs are listed. Each user may be required to sign in to the server with their calendar program account to allow the server to access their calendars.

A reseller system may be provided within the web application executed by the server. The resellers will be referred to as “Paid Subscribers”. The paid subscribers may purchase the components of FIG. 1 and may be granted access to all features of the web application for a monthly subscription fee which will be automatically charged from the user's credit card if the payment processing system allows such. Otherwise the paid subscriber may receive invoices every month, and the paid subscriber can pay the invoice manually as well.

Paid Subscribers will be assigned custom templates which will be custom made based on their existing offline reports. Decisional paragraphs will be included in the report and will be configured by the Paid Subscribers from the templates section.

The server may provide multiple packages for the paid subscribers and these packages will be set in the paid subscriber's profile. Each package will be configurable from the web application where the administrator can create a new package and select what features are available to the paid subscriber under that package. The most basic package may allow the user to enter the address, client information (optional) and notes, and schedule the pick-up/drop-off, but will receive only raw data from the radon testing device. The next package will include the option to generate reports from the web application, plan routes, export state data, etc. The highest package will include the option to bill the paid subscriber's customers, track employee jobs and generate payrolls. The administrator and the paid subscribers may be able to customize the text contained in various SMS notifications as well as emails. For example, the paid subscriber may select a template that matches a “look and feel” or template of their business such that reports generated by the server on behalf of the paid subscriber may contain the logo, trade name, and/or other content associated with the paid subscriber. As a result, the reports generated on behalf of the paid subscriber may appear to be coming from the paid subscriber rather than the system shown in FIG. 1.

The following radon tests may be scheduled by the server and executed by the radon testing device: 1) a Regular 48 Hour Test in which the radon testing device takes 48 readings (one every hour) and averages them; 2) a Side-by-Side Test in which two radon testing devices are placed in close proximity to each other (e.g., 4 inches apart) and each device performs the Regular 48 Hour Test (this may be required for 10% of the tests); and 3) an Extended Test in which the first N hours (selected from 12, 24, 36, or another suitable number of hours) are discarded and an average is calculated for the remaining values measured during the extend of the test. If a window was determined to be open, a door was tampered with, or another determination is made that the testing environment was tampered with during the test, the test may be marked as “invalid” and the measurement data may be discarded. In some embodiments, about 10% of the time a Side-by-Side Test is conducted to find the variance between each radon testing device. It is also preferred to cross-check two radon testing devices that are 4-7 months apart from calibration. For every tenth test that was conducted, or for every tenth percent of individual Paid Subscribers, the server may display a warning to the user that the associated radon testing device is due for a Side-by-Side Test. The calibration factors are received by the server which then applies the factors to the “EPA Protocol Average” to calculate the calibrated or “true” value, thus conforming to the laboratories who calibrate the monitors inside a known radon testing chamber.

In some embodiments, templates of decisional paragraphs may be stored within the server and/or database and may be available to the user to assist the user in generating the report based on the measured radon levels. For example, an acceptable radon level may be 3.9 pCi/L or below, while an unacceptable radon level may be 4.0 pCi/L. Thus, a decisional paragraph may be automatically provided by the server to the user identifying a normal or passing test if the server identifies the average radon level is equal to or below 3.9 pCi/L. Likewise, a decisional paragraph may be automatically provided by the server to the user identifying an elevated or failing test if the server identifies the average radon level is equal to or above 4.0 pCi/L.

Once the user selects multiple jobs and clicks on the “Find my route” button, the server will find the best route and show all locations on a map. The user will have the option to rearrange the jobs and improve the route. The user will also be able to select properties and drop some of them from the route if needed. Once the user presses “Confirm Route”, only then the notifications will be sent to the relevant persons that were set to ON using the toggle switches at the time of adding the order to the server for only those properties that are added to the current route. The user will be shown driving instructions to the user in Google Maps app from property to property.

The server will log each SMS or Email sent by any of the users and may store the log in the database. The log may be displayed in every user's profile. Also, every change in the data contained in the system will also be logged so that in case of any discrepancy the admin can audit the logs. The server will also track the links sent to customers via SMS or Email to determine whether the custom read that message or not.

The firmware for the RS232 adapter will be developed and integrated with the new process flow. The server will have the option to use a “Legacy System” setting for retrieving the measurement data from the radon testing device (e.g., without using the adapter and mobile device). Using this setting, the server will upload the raw data from the radon testing device using a serial cable connected to the radon testing device and using software provided by the radon testing device manufacturer. This will be useful in case the adapter is malfunctioning or not available at the time of generating reports.

While the above features are described as being implemented by the server (i.e., as computer-readable instructions of a web application stored in memory of the server and executed by the server processor), it should be recognized that the above-described features and embodiments may be alternatively or additionally implemented by the application executing on the mobile device and/or by software or firmware operating on the adapter.

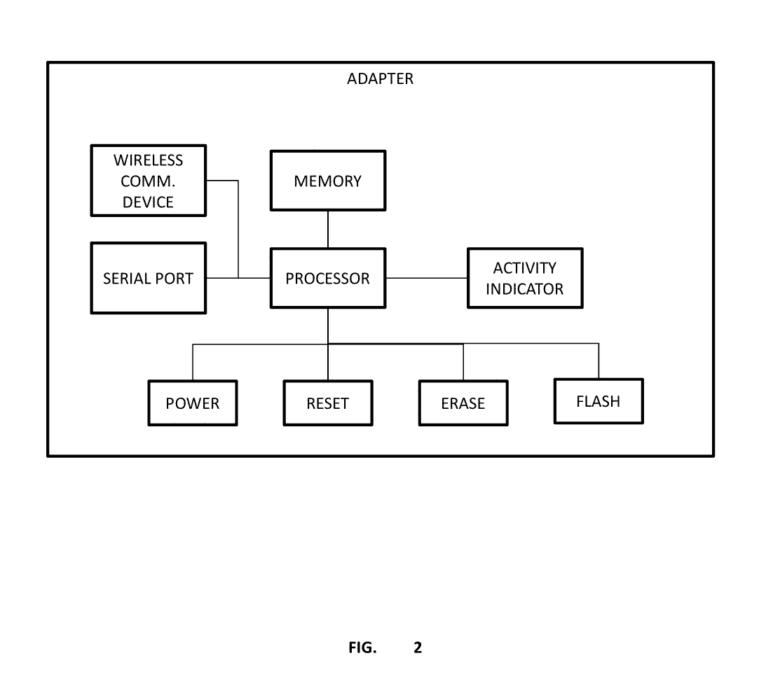

FIG. 2 is a block diagram of an exemplary adapter that may be used with the system shown in FIG. 1.

The adapter includes a plurality of components positioned within a housing. The components include a processor, memory, serial port, and wireless communication device. In one embodiment, the adapter also includes a plurality of buttons or switches, including a power button, a reset button, an erase button, and/or a flash button. The adapter may also include one or more indicators, such as an activity indicator. Each component may be coupled either directly or indirectly to the processor such that the processor may control the operation of each component in some embodiments.

The serial port may include a universal asynchronous receiver/transmitter (UART) that is configured to receive a serial stream of data from the radon testing device. More specifically, the UART may receive the serial stream of measurement data and may interface with the processor to store the measurement data in memory.

The wireless communication device may include an adapter having one or more antennas. The wireless communication device may be configured to communicate with the mobile device using WiFi, Bluetooth, and/or any other suitable protocol.

The power button is a button or switch that the user may press to turn the adapter on or off. The reset button is a button or switch that the user may press to reset or reboot the adapter, thus causing the adapter to re-execute any boot-up code stored in firmware or other memory. The erase button is a button or switch that the user may press to erase the content of the internal memory of the adapter. Specifically, pressing the erase button may cause the adapter to erase all measurement data received from the radon testing device. The flash button is a button or switch that the user may press to download new firmware or other software to the adapter to change the operation of the adapter. In one embodiment, holding the erase button in a depressed state while also pressing the reset button will cause the adapter to erase any wireless connection information (such as credentials, IP address, gateways, etc.) needed to connect to the mobile device and/or the server. This will enable the adapter to be used to connect to a different mobile device and/or server as needed.

The activity indicator may include a light-emitting diode (LED), or any other suitable indicator that may be used by the adapter processor to operate as described herein. In an embodiment in which the activity indicator is an LED, the LED may be configured to emit light in a single color (e.g., red, green, blue, yellow), or may be configured to emit light in one of a plurality of selectable colors. In one embodiment, the activity indicator may emit a green light when the adapter is ready to receive data from the radon testing device. The activity indicator may emit a solid red light that periodically changes to a blue light (or may alternatingly blink red and blue) when the adapter is in the process of receiving measurement data from the radon testing device. When the adapter has completed the receipt of the measurement data from the radon testing device, the activity indicator may emit a solid blue light. When the adapter is uploading the measurement data to the mobile device and thereby to the server, the activity indicator may emit a blinking red light. When the adapter has completed uploading the measurement data to the server, the activity indicator may return to emitting a solid green light. The activity indicator may also emit light in a predefined color and/or pattern when the adapter is connected to the radon testing device, when the adapter is in the process of receiving the measurement data from the radon testing device, and/or during any other suitable activity.

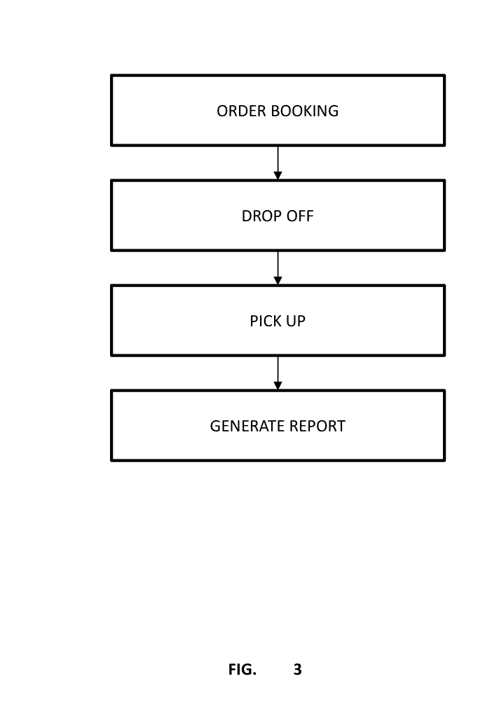

FIG. 3 is a flowchart illustrating a method of generating a radon testing report according to one embodiment. The method is split up into 4 overarching steps in the illustrated embodiment, although it should be recognized that one or more of the steps may be combined together or individual steps may be split apart in some embodiments. The steps may be performed by a user operating the system components described in FIG. 1 on behalf of a client or customer. The user may include a paid subscriber or another provider or operator of the system. The client or customer may be an owner of a building, a real estate agent representing the owner of the building, a tenant of the building, or any other suitable person or entity that may desire to measure the amount of radon within the building.

Step 1: Order Booking

Once the user is contacted for a new request to measure radon in the building (also known as a “booking” or “job”) via phone or email, the user will enter the details of the booking into the server by performing one or more of the following actions:

Enter Address: Start typing the address and the server will show suggestions from Google Maps, for example. The server will split the address into street, unit, city, state and zip at the back-end.

Select Client Type: Click on the type of client this job is for or the person giving the information. Once a client type is selected, its relevant input fields will appear. Data for multiple client types can be filled at the same time.

Enter Client Information: As the user starts typing the name of client, the server will show relevant clients already stored in the server or database. If a client does not exist, the server will give the option to add a new client.

Select Contacts: Select if the report results need to be shared with any existing contact for that client.

Invoice Amount: The amount which user needs to bill to the client. Must be entered whether the invoice is sent immediately or not.

Send Invoice To: Defaulted to “Inspector”. Option to select multiple client types using iPhone-like toggle switches.

Send Non-Interference Agreement To: Defaulted to “Seller”. Option to select multiple client types using iPhone-like toggle switches.

Send Report To: Defaulted to “Buyer”. Option to select multiple client types using iPhone-like toggle switches.

Retest: Defaulted to “No”. Options: “Yes” or “No”.

Mitigation Firm: Only displayed if it is a retest. Takes input just like client, shows you registered firms as you type and the option to add a new one.

Notes: Summary of each job and used to explain to the person completing the other half of the radon test what to do.

Entry Information: Any instructions passed on by the client to enter into the building.

Schedule Drop Off: The server will display a time table for the logged in user from where the user can select a free time slot for drop off (i.e., the setup of the radon testing device and any other equipment needed to perform the radon measurement). If the user is an administrator user, the server will allow the user to view the time table of any other user and schedule the drop off in his/her account. The server will also have a “Strict” checkbox which will lock the time and will not allow the “Plan a route” feature to modify it while making a new route for a user. This option may only be used when the staff performing the drop off needs to be at the drop off location (the building being tested) at the exact time. Notification may be sent to the user half an hour before the scheduled time and at the time when the staff leaves for that building. Alternatively, the notification time may be configurable by the user, and may include 20 minutes, an hour, or any suitable time prior to the scheduled time.

Notify Client On Schedule/Arrival: Defaulted to “Seller”. Option to select multiple client types using iPhone-like toggle switches. This will send a notification to the selected client type once a staff member leaves for the building in Step 2 (Drop Off) below and also once he is at the building.

Upon submission of this form, an invoice will be generated by the server and the client will be sent a link to the invoice on the client device operated by the client. A Non-Interference Agreement will also be generated by the server and its link will be sent to the seller. The seller will need to sign the agreement using a custom DocuSign-like interface which should be compatible with mobile version as well. The server will also send a copy of the agreement to other parties selected above.

Although non-payment of invoice will not stop the process at this step, a notification may be displayed to the client that the report will only be available once the payment has been made.

Step 2: Drop Off

The user will use the “Plan a route” feature to map out the properties and then click on the “Get Directions” button for viewing the driving instructions on Google Maps or Apple Maps application.

If “Notify Client” is selected, the system will send an SMS to the person that we are on our way to their building and will be reaching in approximately X minutes. The time will be calculated using Google Maps API. A link to representative's contact card/ID will be included in the text message showing his name, picture and state license number. The link will be tracked and will store timestamps when it is clicked so that we know that the user has read that message.

Once the user has reached the building, he will be shown “Entry Information” and “Notes” stored at the time of order booking. Contact information of inspector, listing agent, seller, buyer agent and buyer will also be shown at this point so that in case the staff needs to contact them he could click on their number and call them.

The fields for this step are defined below and may be displayed, modified, and/or filled in via a mobile device operated by the inspector or other person performing the drop off:

Define Rooms: The user will add rooms and enter details as defined in entities section. Multiple rooms can be added.

Add Monitors: For each room, the user will add a monitor and define its placement as defined in entities section. If two monitors are added to the same room, we will place a toggle switch for “Side by Side” test. There will also be a toggle switch to include Appendix E to the report and by default it will not be included.

Select Property Photo: Based on the property address (e.g., building address), system will fetch building images using Google Custom Search API and allow the user to select a photo from the list.

Choose Floor Plan: The user will upload the floor plan created on his tablet/phone.

Foundation Type: The user will select the type of foundation.

Foundation Height: This will be displayed only when needed.

Home Status: Defaulted to “Occupied”. Other options: “Vacant”.

Lock Box: Defaulted to “None”. Other options: “Sentrilock”, “Combo Push”, “Combo Roll”

Re-entry: Defaulted to “Confirmed”. Other options: “Not Confirmed”, “Agent Accompany”, “Seller will be home”

Vent Status: Defaulted to “Open”

Vents: Defaulted to “Fireplace”

Schedule Pick Up: The system will display a time table for the logged in user from where the user can select a free time slot for pick up (at least 48 hours from drop-off time). If it's an admin user, the system will allow to view time table of any other user and schedule it in his/her account. Notification will be sent to user half an hour before the scheduled time and at the time when staff leaves for that building.

Step 3: Pick Up

Just like drop off, the “Plan a route” and “Notify Client” features will be available in this step as well.

View Raw Data: The raw measurement data will be retrieved from the radon testing device using the RS232 adapter and the user can view the data in a new window at this stage to make sure everything is in place.

Extended: Defaulted to “No”. If the test was extended, the user will select “12 Hours”, “24 Hours” or “36 Hours” from the options.

House Opened: Defaulted to “No”. Other options: “Tampered”, “Open Window/Door”, “Compromised”, “Other”

Fault: This will be displayed only when house was opened. It will be defaulted to “None” however the user can select any fault if it exists.

If the layout information was not filled at the time of drop off, the user will be required to fill that information. A “Layout Missing” tag will also be shown with the building details in such a case.

Step 4: Generate Report

Retest Information: Shown only if it is a retest. Defaulted to “Included” but if “Excluded” is selected the report will be sent as a fresh report.

Missing Information: Any information that was not filled in the previous steps will be asked again in this step.

Reaffirm Drop Off and Pick Up times: In case the pick-up time is less than 48 hours from the drop off time, the user will need to add missing data points and make sure that the report shows at least 48 hours of data.

Once this form is submitted, the system will generate the report and the link to the report will be sent to the client. Upon clicking the link, if the client has already paid, we will ask him to agree to the terms to limit our liability. If the payment is pending, he will be taken to the invoice payment page so that he can enter his credit card details and pay for the invoice.

There will be cases when the inspector does not want to disclose the buyer information and wants to forward the report to the buyer himself instead. For this we will have a “Send buyer report link to inspector” toggle switch while placing the order. If this is turned ON, the inspector will receive two emails, one for himself (which will show the paid amount as well) and one for his client/buyer.

Although specific features of various embodiments of the disclosure may be shown in some drawings and not in others, this is for convenience only. In accordance with the principles of the disclosure, any feature of a drawing or other embodiment may be referenced and/or claimed in combination with any feature of any other drawing or embodiment.

This written description uses examples to describe embodiments of the disclosure and also to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system for monitoring a level of gas, the system comprising:
a gas testing device including:
at least one sensor configured to collect a measurement of an amount of gas in a location,
a processor configured to:
receive a signal from the at least one sensor, wherein the signal includes the measurement,
generate data based on the signal, and
store the data in a memory;
an adapter coupled to the gas testing device by a serial port of the gas testing device and comprising an adapter processor and an adapter memory, wherein the adapter processor is configured to receive the data from the gas testing device, the adapter processor further configured to store the data in the adapter memory;
a mobile device coupled to the adapter and configured to receive the data from the adapter; and
a server coupled to the mobile device via a network and comprising a server processor and a server memory, wherein the server processor is configured to receive the data from the mobile device, the server processor further configured to store the data in the server memory.

2. A method for monitoring a level of gas, the method comprising:
collecting, by at least one sensor of a gas testing device, a measurement of an amount of gas in a location;
receiving, by a processor of the gas testing device, a signal from the at least one sensor, wherein the signal includes the measurement,
generating, by the processor of the gas testing device, data based on the signal;
storing, by the processor of the gas testing device, the data in a memory;
receiving, by an adapter processor of an adapter coupled to the gas testing device by a serial port of the gas testing device, the data from the gas testing device;
storing, by the adapter processor, the data in an adapter memory of the adapter;
receiving, by a mobile device coupled to the adapter, the data from the adapter;
receiving, by a server processor of a server coupled to the mobile device via a network, the data from the mobile device; and
storing, by the server processor, the data in a server memory of the server.
